(19) United States
(12) Patent Application Publication
(10) Pub. No.: US 2015/0165069 A1
(43) Pub. Date: Jun. 18, 2015

(54) Nanoparticles for Targeting for a Biological Application

(71) Applicant: CAPSUM, Marseille (FR)

(72) Inventors: Nicolas Atrux-Tallau, Le Bourg D'oisans (FR); Thomas Delmas, Marseille (FR); Mathieu Goutayer, Saint Malo (FR); Audrey Royere, Paris (FR)

(21) Appl. No.: 14/403,626

(22) PCT Filed: May 28, 2013
(86) PCT No.: PCT/EP2013/060966

§ 371 Date: Nov. 25, 2014

(30) Foreign Application Priority Data
May 29, 2012 (FR) 12 54920

(51) Int. Cl. A61K 47/48; A61K 31/343; A61K 47/34

(52) U.S. Cl. CPC A61K 47/48869; A61K 47/34; A61K 47/48346; A61K 47/4813; A61K 47/4823; A61K 31/343

(57) ABSTRACT

This invention concerns a nanoparticle comprising:
a core consisting of a lipid phase (L₁) or an aqueous phase (A₁);
at least one surfactant comprising a hydrophilic part and a lipophilic part;
an internal membrane surrounding the core;
an external membrane surrounding the internal membrane; and
at least one targeting ligand comprising a lipophilic part and a hydrophilic part.

This invention concerns targeted nanoparticles for a biological application.

Currently, the use of nanoparticles for the vectorisation of active ingredients is of significant interest because they are a promising solution in terms of improving the efficacy of active ingredients, whether in the field of cosmetics, dermatological pharmaceuticals, or pharmaceuticals.

In order to ensure drug delivery at their biological site of action, it is necessary to target the nanoparticles in which they are encapsulated at the site of interest. To this end, it is known to provide these nanoparticles with targeting ligands allowing for promotion of interactions between the nanoparticle and the biological medium targeted.

These targeting ligands are generally grafted onto nanoparticles following their production, necessitating a chemical step of synthesis on the surface of the particles and, frequently, the use of solvents and/or additional purification steps. This approach is thus costly in terms of time and material and human resources, and also necessitates very strict quality control for the final product.

Additionally, these nanoparticles generally have on their surface a crown of molecular chains that may have various functions, in particular that of stabilising the nanoparticles. Thus, the targeting ligands are generally grafted onto the end of these chains in order to expose them to the surface of the crown rather than embedding them on the inside, which entails additional constraints during the manufacture of the nanoparticles.

Accordingly, it would be of particular interest to be able to provide nanoparticles with targeting properties without being required to position the targeting ligands on the surface of the nanoparticles by grafting and to have a simpler, less expensive method allowing for the preparation of such targeting nanoparticles.

This invention seeks to provide nanoparticles having targeting properties due to the presence of targeting ligands that are not located on their surface.

This invention further seeks to provide a method for preparing these nanoparticles that allows them to be provided with targeting properties simply and at a reduced cost.

Thus, this invention concerns a nanoparticle comprising:
a core consisting of a lipid phase (L₁) or an aqueous phase (A₁);
at least one surfactant comprising a hydrophilic part and a lipophilic part;
an internal membrane surrounding the core;
an external membrane surrounding the internal membrane; and
at least one targeting ligand comprising a lipophilic part and a hydrophilic part;

in which:
when the core consists of a lipid phase (L₁):
   the internal membrane constitutes a lipid phase (L₂) comprising the lipophilic part of the surfactant;
   the external membrane constitutes an aqueous phase (A₂) comprising the hydrophilic part of the surfactant; and
   the targeting ligand is such that its lipophilic part is in the lipid phase (L₂) and its hydrophilic part has a length that is less than the thickness of the external membrane in the aqueous phase (A₂);
when the core consists of an aqueous phase (A₁):
   the internal membrane constitutes an aqueous phase (A′₂) comprising the hydrophilic part of the surfactant; and
   the external membrane constitutes a lipid phase (L′₂) comprising the lipophilic part of the surfactant;

Thus, this invention concerns a nanoparticle comprising:
a core consisting of a lipid phase (L₁):
at least one surfactant comprising a hydrophilic part and a lipophilic part;
an internal membrane surrounding the core and constituting a lipid phase (L₂) comprising the lipophilic part of the surfactant;
an external membrane surrounding the internal membrane and constituting an aqueous phase (A₂) comprising the hydrophilic part of the surfactant; and
at least one targeting ligand comprising a lipophilic part and a hydrophilic part, whereby the lipophilic part is in the lipid phase (L₂) and the hydrophilic part has a length that is less than the thickness of the external membrane in the aqueous phase (A₂).

According to one embodiment, the external membrane that constitutes an aqueous phase (A₂) is between 1 and 7 nm in length, advantageously between 1.5 and 6 nm, preferably between 2 and 5 nm, and the hydrophilic part of the targeting ligand located in the aqueous phase (A₂) has a length between 0.2 and 5 nm, advantageously between 0.5 and 4 nm, preferably between 0.5 and 3 nm.

This invention additionally concerns a nanoparticle comprising:
a core consisting of an aqueous phase (A₁):
at least one surfactant comprising a hydrophilic part and a lipophilic part;
an internal membrane surrounding the core and constituting an aqueous phase (A′₂) comprising the hydrophilic part of the surfactant;
an external membrane surrounding the internal membrane and constituting a lipid phase (L′₂) comprising the hydrophilic part of the surfactant; and
at least one targeting ligand comprising a lipophilic part and a hydrophilic part.

Thus, when the core consists of a lipid phase (L₁), the internal membrane constitutes a lipid phase (L₂), the external membrane constitutes an aqueous phase (A₂), and the nanoparticle is considered a ‘lipid nanoparticle’ because it consists essentially of lipids.

When the core consists of an aqueous phase (A₁), the internal membrane constitutes an aqueous phase (A′₂), the external membrane constitutes a lipid phase (L′₂), and the nanoparticle is considered an ‘aqueous nanoparticle’ because it consists essentially of water.

In the context of this description, ‘nanoparticle’ refers to an assembly of atoms in which at least one of the three dimensions is on the nano scale. More specifically, this refers to objects having a size of 10 to 1000 nm.

According to the invention, the nanoparticle comprises a core consisting of a lipid phase (L₁) or an aqueous phase (A₁).

In this description, ‘lipid phase’ refers to a phase having the property of solubilising apolar compounds such as lipids, fat, and oils.

Within the meaning of this invention, ‘lipid’ refers to all fats or substances containing fatty acids present in animal fats and vegetable oils. They are small hydropholic or amphiphilic molecules consisting principally of carbon, hydrogen, and oxygen and having a density less than that of water. The lipids may be present in the solid state, as with waxes, or the liquid state, as with oils.

Additionally, ‘aqueous phase’ refers to a phase comprising water and having the property of solubilising polar compounds.

The nanoparticle further comprises one or more surfactants.

In this description, ‘surfactant’ refers to an amphiphilic molecule having two parts with different polarities, one of which is lipophilic and apolar and the other is hydrophilic and polar. A surfactant may be ionic (cationic or anionic), zwitterionic, or non-ionic.

Within the meaning of this invention, ‘hydrophilic’ structures are chemical structures having an affinity for water. If, additionally, this structure may dissolve in water, it is described as ‘water-soluble’.

Additionally, ‘lipophilic’ refers to a chemical structure having an affinity for organic solvents and lipids (oils and/or waxes) and avoiding contact with a polar solvent such as water. A lipophilic compound that is soluble in lipids is described as ‘lipid-soluble’.

The surfactant is advantageously an anionic surfactant, a non-ionic surfactant, a cationic surfactant, or a mixture thereof. The molecular mass of the surfactant is between 150 g/mol and 10000 g/mol, advantageously between 250 g/mol and 1500 g/mol.

If the surfactant is an anionic surfactant, it is selected from the group of alkylsulphates, alkylsulphonates, alkylarylsulphonates, alkaline alkylphosphates, dialkylsulphosuccinates, and alkaline earth salts of saturated or unsaturated fatty acids. These surfactants advantageously have at least one hydrophobic hydrocarbon chain having a number of carbon atoms greater than 5, or 10, and at least one hydrophilic anionic group such as a sulphate, sulphonate, or carboxylate linked to one end of the hydrophobic chain.

If the surfactant is a cationic surfactant, it is selected, e.g., from the group of an alkylpyridium halide or alkylammonium salt such as n-ethyldodecylammonium chloride or bromide, or cetylammonium bromide (CTAB). These surfactants advantageously have at least one hydrophobic hydrocarbon chain having a number of carbon atoms greater than 5, or 10, and at least one hydrophilic cationic group a quaternary ammonium cation.

If the surfactant is a non-ionic surfactant, it is selected, e.g., from polyoxyethylenated and/or polyoxypropylenated derivatives of fat alcohols, fatty acids, or alkylphenols, arylphenols, or from glycoside alkyls, polysorbates, cocamides, and saccharose esters.

Preferably, the surfactants present in the nanoparticle are selected from non-ionic surfactants comprising a long polymer chain of the polyethylene oxide (PEG) type. These chains are positioned on the surface of the nanoparticle and allow for it to be stabilised.

The surfactants may also be selected from the amphiphilic lipids.

Amphiphilic lipids include a hydrophilic part and a lipophilic part. They are generally selected from compounds in which the lipophilic part comprises a saturated or unsaturated, linear or branched chain having 8 to 30 carbon atoms. They may be selected from the phospholipids, cholesterols, lysolipides, sphingomyelins, tocopherols, stearylamine glucolipids, cardiolipins of natural or synthetic origin; molecule consisting of a fatty acid coupled with a hydrophilic group by an ether or ester function such as sorbitan esters, e.g., sorbitan monooleates and monolaurates sold under the names Span® by ICI; polymerised lipids; lipids conjugated with short polyethylene oxide (PEG) chains such as the non-ionic surfactants sold under the trade name Tween® by ICI Americas, Inc. And Triton X-100®, marketed by Union Carbide Corp.; sugar esters such as saccharose mono- and di-laurates, mono- and di-palmitates, mono- and distearates; whereby the surfactants may be used alone or in mixtures such as Cosbiol® from Laserson.

The content by mass of surfactant is, e.g., from 1 to 60%, advantageously from 5 to 50%, preferably from 10 to 40% of the total weight of the nanoparticle.

According to the invention, the nanoparticle further comprises an internal membrane surrounding the core:
if the core consists of a lipid phase (L₁), the internal membrane constitutes a lipid phase (L₂) comprising the lipophilic part of the surfactant; and
if the core consists of an aqueous phase (A₁), the internal membrane constitutes an aqueous phase (A′₂) comprising the hydrophilic part of the surfactant.

Within the meaning of this invention, ‘surround’ refers to completely covering. This term is interchangeable with ‘encapsulate’.

Thus, the internal membrane completely covers the external surface of the core.

The nanoparticle further comprises an external membrane surrounding the internal membrane:
if the core consists of a lipid phase (L₁), the external membrane constitutes an aqueous phase (A₂) comprising the hydrophilic part of the surfactant; and
if the core consists of an aqueous phase (A₁), the external membrane constitutes a lipid phase (L′₂) comprising the lipophilic part of the surfactant.

Thus, the external membrane completely covers the external surface of the internal membrane.

The external membrane may also be referred to as the ‘crown’.

As noted above, according to one embodiment, when the core consists of a lipid phase (L₁), the external membrane constituting an aqueous phase (A₂) has a thickness between 1 and 7 nm, advantageously between 1.5 and 6 nm, preferably between 2 and 5 nm.

The thickness of the external membrane is measured by small angle neutron scattering (SANS).

By manipulating the composition of the continuous phase in which the nanoparticles are dispersed in terms of an H₂O/D₂O mixture, it is possible to measure the size of the nanoparticle on the one hand and the size of the nanoparticle without the crown on the other, thus cancelling out the difference between the external continuous phase and the crown. Accordingly, a measurement of the thickness of the crown can be extracted from this:

e=R(nanoparticle)−R(nanoparticle without crown)

If the membrane—internal or external—constitutes a lipid phase (L₂) or (L′₂), it consists essentially of the lipophilic parts of the surfactants, in particular the lipid-soluble surfactants.

In this description, ‘lipid-soluble surfactant’ refers to a surfactant in which the lipophilic part is longer than the hydrophilic part, thus making it lipid-soluble

According to one embodiment, the lipid-soluble surfactants are phospholipids. Phospholipids are amphiphilic lipids having a phosphate group, in particular phosphoglycerides. They most frequently include a hydrophilic end consisting of the phosphate group, which may be substituted, which will be positioned spontaneously in the aqueous phase (A₂) or (A′₂) and two hydrophobic ends consisting of fatty acid chains, which will be positioned spontaneously in the lipid phase (L₂) or (L′₂).

Phospholipids include phosphatidyl choline, phosphatidyl ethanolamine, phosphatidyl inositol, phosphatidyl serine, and sphingomyelin.

If the membrane—internal or external—constitutes an aqueous phase (A₂) or (A′₂), it consists essentially of the hydrophilic parts of the surfactants, in particular the water-soluble surfactants.

In this description, ‘water-soluble surfactant’ refers to a surfactant in which the hydrophilic part is longer than the lipophilic part, thus making it water-soluble.

The water-soluble surfactants are preferably alkoxylated and preferably include at least one hydrophilic chain consisting of ethylene oxide (PEO or PEG) or ethylene oxide and propylene oxide patterns. Preferably, the number of these patterns in the chain is between 2 and 500, whereby the hydrophobic part preferably comprises fatty acids having a number of carbon atoms between 6 and 50.

Examples of surfactants include, in particular, conjugated polyethylene glycol/phosphatidyl-ethanolamine (PEG-PE) compounds, fatty acid and polyethylene glycol ethers such as those sold under the trade name Brij® (e.g., Brij® 35, 58, 78, or 98) by ICI Americas Inc., fatty acid and polyethylene glycol esters such as those sold under the trade name Myrj® by Croda (e.g., Myrj® S 20, 40, 50, or 100), and ethylene oxide and propylene oxide block coplymers sold under the trade name Pluronic® by BASF AG (e.g., Pluronic® F68, F127, L64, L61, 10R4, 17R2, 17R4, 25R2, or 25R4), or those sold under the trade name Synperonic® by Unichema Chemie BV (e.g., Synperonic® PE/F68, PE/L61, or PE/L64).

Other examples include APG (alkyl polyglycoside), alkyl polyglycerols, and saccharose esters.

According to one embodiment, the hydrophilic part of the water-soluble surfactants consists of polyethylene glycol (PEG) chains. These PEG chains create a steric gene allowing for the prevention of the coalescence of the nanoparticles, thus stabilising them. Additionally, these compounds may give the nanoparticle a stealth property by deceiving the immune defences of the body.

According to the invention, the nanoparticle comprises at least one targeting ligand comprising a lipophilic part and a hydrophilic part, such that, when the core consists of a lipid phase (L₁), the lipophilic part is in the lipid phase (L₂), and the hydrophilic part has a length that is less than the thickness of the external membrane in the aqueous phase (A₂).

As noted above, according to one embodiment, when the core consists of a lipid phase (L₁), the hydrophilic part of the targeting ligand located in the aqueous phase (A₂) has a length between 0.2 and 5 nm, advantageously between 0.5 and 4 nm, preferably between 0.5 and 3 nm.

In this description, ‘targeting ligand’ refers to a molecule having a specific interaction with another compound, such as a receptor present on the surface of the cell or target tissue.

‘Specific’ refers to the fact that the ligand establishes a substantially stronger bond with the target cell or tissue than with non-targeted cells and tissues.

A targeting ligand is, e.g., an antibody, peptide, saccharide, aptamere, oligonucleotide, or peptidomimetic.

The targeting ligand may also be referred to as ‘targeting molecule’.

In the case of an aqueous nanoparticle, the hydrophilic part of the targeting ligand, which is typically the part allowing for the targeting of the biological sites of interest, is embedded within the internal membrane and thus not exposed to the surface of the nanoparticle.

In the case of a lipid nanoparticle, the length of its hydrophilic part and the thickness of the external membrane mean that the external end of the targeting ligand is located within the external membrane and not exposed to the surface of the nanoparticle, unlike prior-art nanoparticles with targeting ligands.

In fact, application FR2935001, for example, describes oil-in-water fluorescent emulsions in which the oil droplets are stabilised by a surfactant layer, which may comprise a targeting agent. This comprises an amphiphilified grafting co-surfactant, the hydrophilic part of which is bonded to a biological ligand positioned on the surface of the droplets.

Surprisingly, the fact that the targeting ligand is not exposed to the surface of the nanoparticle does not prevent cellular targeting. The targeting ligand thus allows the nanoparticles according to the invention to better target biological sites of interest than nanoparticles without such ligands, as shown in detail in the examples.

According to one embodiment, the nanoparticle comprises at least one active ingredient.

In this description, ‘active ingredient’ refers to a compound having a beneficial physiological effect on the element in question. This includes, for example, protecting, maintaining, caring for, healing, perfuming, flavouring, or colouring.

The active ingredient is advantageously a cosmetic, dermatological pharmaceutical, or pharmaceutical.

The nanoparticle may contain the active ingredient in the form of a pure liquid or a solution of the active ingredient in a liquid solvent, or a dispersion of the active ingredient in a liquid. It may also be molecularly dispersed in the core, be in the form of microcrystals, or in the form of amorphous aggregates.

Within the meaning of this invention, ‘molecularly dispersed in the core’ refers to the fact of being solubilised in the form of molecules isolated in the core.

A lipophilic active ingredient is preferably incorporated in a lipid nanoparticle, whilst a hydrophilic active ingredient is preferably incorporated in an aqueous nanoparticle.

If the active ingredient is a cosmetic, it may be selected from sodium hyaluronate or other hydrating/repairing molecules, vitamins, enzymes, anti-wrinkle, anti-aging agents, protectants/anti-free radical agents, antioxidants, soothing, softening agents, anti-irritants, tensors/smoothers, emollients, thinning agents, anti-sponginess agents, firming agents, sheathing agents, draining agents, anti-inflammatories, depigmenting agents, whiteners, self-tanners, exfoliants, stimulating cellular renewal or cutaneous microcirculation, absorbing or filtering UV, anti-dandruff agents.

A cosmetic is cited, e.g., in Directive 93/35/EEC of the Council dated 14 Jun. 1993. This product is, e.g., a cream, emulsion, lotion, gel, and oil for the skin (hands, face, feet, etc.), a foundation (liquid, paste), preparation for baths and showers (salts, foams, oils, gels, etc.), a hair care agent (hair dyes and bleaches), a cleaning product (lotions, powders, shampoos), a hair maintenance product (lotions, creams, oils), a hair styling product (lotions, hairsprays, brilliantines), a product for application to the lips, a sun protection product, a sunless tanning product, a product for skin whitening, an anti-wrinkle product.

Dermatological pharmaceuticals refer more specifically to agents acting on the skin.

If the active ingredient is a pharmaceutical, it is advantageously selected from anticoagulants, anti-thrombogenics, anti-mitotics, anti-proliferation agents, antiadhesives, anti-migration agents, cellular adhesion promoters, growth factors, anti-parasitic molecules, anti-inflammatories, angiogenics, angiogenesis inhibitors, vitamins, hormones, proteins, antifungals, antimicrobials, antiseptics, or antibiotics.

The targeting ligand may also be an active ingredient as defined above.

Preferably, the nanoparticles have a diameter between 10 and 1000 nm, advantageously between 20 and 200 nm.

The size of the nanoparticles is measured by light diffusion. For example, a Zeta Sizer Nano ZS (Malvern Instrument) is used. The principle is based on a measurement of the characteristic diffusion time of the particles by brownian movement in order to deduce their size. This method is described, in particular, by the supplier of the measurement device used: http://www.malverninstruments.fr/labfre/products/zetasizer/zetasizer_nano/zetasizer_nano_zs.htm.

According to one embodiment, the nanoparticles are solid lipid nanoparticles, micelles, or liposomes.

In this description, ‘solid lipid nanoparticle’ refers to a nanoparticle in which the lipids are solid.

In this description, ‘micelle’ refers to a spheroid aggregate of amphiphilic molecules having a hydrophilic polar head and a hydrophobic chain that is formed when the amphiphilic molecule concentration exceeds a certain threshold known as the critical micellar concentration (CMC).

More specifically, micelle is ‘direct’ is the continuous phase in which the nanoparticle is located is polar, such as water, because the molecules have their hydrophilic part on the surface, and their hydrophobic part in the core of the micelle. On the other hand, a micelle is ‘inverse’ if the continuous phase is apolar, such as oil, because the hydrophobic parts are on the outside. The nanoparticles according to the invention are, e.g., direct micelles.

In this description, ‘liposome’ refers to an artificial vesicle consisting of concentric lipid bilayers containing aqueous compartments. The liposomes are generally obtained with amphiphilic lipids such as phospholipids.

According to one embodiment, the nanoparticle is placed in continuous phase and forms a nanoemulsion with it.

In this description, a ‘nanoemulsion’ is a composition having at least one lipid phase and at least one aqueous phase, whereby one of the two phases is the dispersed phase and the other is the continuous phase, in which the average droplet size of the dispersed phase is less than 1 μm, advantageously between 10 and 500 nm, and for which the lipids are in the liquid state.

If the nanoparticle is lipid, the continuous phase is aqueous and the nanoemulsion is referred to as a ‘direct nanoemulsion’.

If the nanoparticle is aqueous, the continuous phase is lipid and the nanoemulsion is referred to as an ‘inverse nanoemulsion’.

The continuous phase of the nanoemulsion may comprise an active ingredient.

It is thus possible to combine initially incompatible active ingredients by incorporating a lipophilic active ingredient in the lipid nanoparticle and a hydrophilic active ingredient in the continuous aqueous phase in a direct nanoemulsion.

In the case of a direct nanoemulsion, the lipid phase (L₁) constituting the core may comprise solubilised lipid-soluble surfactants, which may be in the form of micelles, and the continuous aqueous phase may comprise solubilised water-soluble surfactants, which may be in the form of micelles.

In the case of an inverse nanoemulsion, the aqueous phase (A₁) constituting the core may comprise solubilised water-soluble surfactants, which may be in the form of micelles, and the continuous lipid phase may comprise solubilised lipid-soluble surfactants, which may be in the form of micelles.

According to one embodiment, the lipid phase (L₁) and/or the lipid phase (L₂) or (L′₂) of the nanoparticles comprises at least one active ingredient as defined above, in particular a cosmetic, dermatological pharmaceutical, or pharmaceutical.

The active ingredient may thus only be present in the lipid phase (L₁).

The active ingredient may also only be in the lipid phase (L₂) or (L′₂).

Lastly, the active ingredient may be present in each of the two lipid phases (L₁) and (L₂) or (L′₂), in which case it may be identical or different from one phase to the other.

The active ingredient may be in the form of a single active ingredient or a mixture of several active ingredients.

According to one embodiment, the aqueous phase (A₁) and/or the aqueous phase (A₂) or (A′₂) of the nanoparticles comprises at least one active ingredient as defined above, in particular a cosmetic, dermatological pharmaceutical, or pharmaceutical.

The active ingredient may thus only be present in the aqueous phase (A₁).

The active ingredient may also only be in the aqueous phase (A₂) or (A′₂).

Lastly, the active ingredient may be present in each of the two aqueous phases (A₁) and (A₂) or (A′₂), in which case it may be identical or different from one phase to the other.

The active ingredient may be in the form of a single active ingredient or a mixture of several active ingredients.

According to one embodiment, the targeting ligand is also an active ingredient as defined above. In this particular case, the active ingredient is present simultaneously in the lipid phase (L₂) or (L′₂) and the aqueous phase (A₂) or (A′₂), respectively.

More generally, if the active ingredient is amphiphilic, its lipophilic part will be positioned in the lipid phase (L₂) or (L′₂), and its hydrophilic part in the aqueous phase (A₂) or (A′₂); thus, the active ingredient will be present in both phases.

According to one embodiment, the lipid phase (L₁) and/or the lipid phase (L₂) or (L′₂) comprises at least one solubilising lipid.

The solubilising liquid may thus only be present in the lipid phase (L₁).

The solubilising lipid may also only be in the lipid phase (L₂) or (L′₂).

Lastly, the solubilising lipid may be present in each of the two lipid phases (L₁) and (L₂) or (L′₂), in which case it may be identical or different from one phase to the other.

The solubilising lipid may be in the form of a single solubilising lipid or a mixture of several solubilising lipids.

In this description, ‘solubilising lipid’ refers to a lipid having an affinity with another lipid sufficient to allow for solubilisation.

The solubilising lipid used is advantageously selected based on the lipids and/or active ingredients to solubilise. It also generally has a close chemical structure in order to ensure the desired solubilisation. It may be an oil or a wax. Preferably, the solubilising lipid is solid at room temperature (20° C.), but liquid at body temperature (37° C.).

If the lipid to be solubilised is an amphiphilic liquid of the phospholipid type, the solubilising lipid may be selected from glycerol derivatives, in particular glycerides obtained by esterification of glycerol with fatty acids.

The preferred solubilising lipids, in particular for phospholipids, are fatty acid glycerides, in particular saturated fatty acids, in particular saturated fatty acids comprising from 8 to 10 carbon atoms, advantageously from 12 to 18 carbon atoms. Preferably, it is a mixture of different glycerides (mono-, di-, and/or triglycerides).

Preferably, these are glycerides of saturated fatty acids comprising from 0% to 20% by weight of C8 fatty acids, from 0% to 20% by weight of C10 fatty acids, from 10% to 70% by weight of C12 fatty acids, and from 5% to 30% by weight of C18 fatty acids.

More specifically, mixtures of semi-synthetic glycerides are preferred that are solid at room temperature and sold under the trade name Suppocire® NC or Lipocire™ by Gattefosse and approved for injection into humans. Type N Suppocire® products are obtained by direct esterification of fatty acids and glycerol. These are semi-synthetic glycerides of C8-C18 saturated fatty acids; thus, the quali-quantitative composition is indicated in the table below.

The quantity of solubilising lipid may vary widely depending on the nature and quantity of amphiphilic lipid present in the lipid phase(s). Generally, the content by mass of solubilising lipid is from 1 to 99%, advantageously from 5 to 80%, preferably from 40 to 75% of the total weight of the lipid phase.

### Fatty Acid Composition of Suppocire® NC from Gattefossé

| Chain length | % by weight |
|---|---|
| C8 | 0.1-0.9 |
| C10 | 0.1-0.9 |
| C12 | 25-50 |
| C14 | 10-24.9 |
| C16 | 10-24.9 |
| C18 | 10-24.9 |

The solubilising lipid may also be chosen from oils.

The oils used preferably have a hydrophilic-lipophilic balance (HLB) lower than 8 and, more preferably, between 3 and 6. Advantageously, the oils are used without chemical or physical modifications prior to the formation of the emulsion.

Depending on the intended application, the oils may be selected from the group of biocompatible oils, in particular oils of natural (vegetable or animal) or synthetic origin. Examples of such oils include natural plant oils, in particular soya, flaxseed, palm, peanut, olive, grapeseed, and sunflower seed oil; examples of synthetic oils include, in particular, triglycerides, diglycerides, and monoglycerides. These oils may be first press, refined, or inter-esterified.

Various excipients may be added either to the composition of the nanoparticle itself or the continuous phase, if the nanoparticle is contained in a nanoemulsion. These excipients may be of different types, in particular colourants, scents, fragrances, stabilisers, preservatives, emulsifiers, thickeners, or other active ingredients in an appropriate quantity.

Preferably, in the case of a direct nanoemulsion, the fragrances are added to the lipid phase (L₁) and the colourants to the continuous aqueous phase.

The targeting ligand of the nanoparticle according to the invention must be able to position itself at the interface of the internal and external membranes of the nanoparticles, and must therefore have a certain amphiphilic nature.

The targeting ligand is preferably selected from the compounds of formula (I):

A-Y-B (I)

in which:
A is the lipophilic part;
Y is a chemical group capable of linking A and B via covalent bonds; and
B is the hydrophilic part.

The lipophilic part A of the targeting ligand allows it to anchor in the lipid phase (L₂) or (L′₂) of the nanoparticle. It may comprise, in particular, an a linear or branched, saturated or unsaturated C₁₆-C₁₈ alkyl chain.

According to one embodiment, the covalent bonds resulting from the presence of the Y group and affixing A to B arise from the reaction between one chemical function initially carried by A before its reaction with B and a complementary chemical function carried by B before their reaction with A. By way of example only, examples of covalent bonds arising from the reaction include the following:
from an amine and an ester activated, e.g., by an N-succinimidyl group resulting in the formation of amide bonds;
from an oxyamine and an aldehyde resulting in the formation of oxime bonds; and
from a maleimide and a thiol resulting in the formation of thioether bonds.

The hydrophilic part B of the targeting ligand allows it to anchor in the aqueous phase (A₂) or (A′₂) of the nanoparticle.

In the case of a lipid nanoparticle, the length of the hydrophilic part B is such that the end of the targeting ligand is located in the external membrane and not beyond the surface of this membrane.

The amphiphilic nature of the targeting ligand may be evaluated using its Log P value.

Preferably, the targeting ligand has a Log P value between −4 and 4, advantageously between 2.5 and 2.5, preferably between −1.5 and 1.5.

The Log P value is generally measured by the ‘shaken flask’ method. This method consists of solubilising a known quantity of solute in a known volume of octanol and water. The biphasic solution is then shaken until equilibrium (t>1 h), and then the distribution of the solute is measured in each solvent. Generally, this quantification of the solute concentrations in each phase is carried out by UV/visible spectroscopy. The Log P is then obtained by the following formula:

Log P=log(concentration of solute in octanol/concentration of solute in water)

The targeting ligand is, e.g., a sugar, biomolecule, polymer, or biopolymer. These molecules may also be ‘lipidised’, i.e., provided with a more lipophilic character by grafting a carbonated chain. This carbonated chain is C2-C18, advantageously C6-C18.

In this description, ‘sugar’ refers to any family of chemical molecules close to saccharose, belonging to the class of carbohydrates. These include saccharose, glucose, and fructose.

In this description, ‘biomolecule’ refers to a molecule involved in the metabolic process and the maintenance of a living organism, e.g., carbohydrates, lipids, proteins, water, and nucleic acids. Thus, they consist mainly of carbon, hydrogen, oxygen, nitrogen, sulphur, and phosphorus. ‘Biomolecule’ also refers to molecules identical to those found in vivo, but obtained by other means.

Thus, ‘biopolymer’ refers to a polymer that is also a biomolecule.

Advantageously, the targeting ligand is a biomolecule selected from the group of peptides, proteins, and enzymes.

According to one variant, the targeting ligand is a lipidised peptide such as a palmitoyl peptide, acetyl peptide, or undecenoyl peptide.

Thus, in the case of lipidised peptides, the lipid character arises from grafting on the peptide of a lipid such as a fatty acid, and, in particular, acetic or palmitic acid.

According to another variant, the targeting ligand is a polysaccharide such as hyaluronic acid, chitosan, or dextran.

Advantageously, the ligand is not grafted, coupled, conjugated, or bonded in any way with another compound.

In this description, ‘compound (X) grafted to a compound (Y)’ refers to the fact that the compound (X) has one or more chemical groups that have interacted with one or more chemical groups of the compound (Y), thus resulting in the formation of bonds, e.g., covalent, between the compound (X) and the compound (Y). This formation of bonds may thus be described as grafting, coupling, or conjugation.

Advantageously, the targeting ligand is a cosmetic active ingredient as defined above.

Preferably, the targeting ligand is selected from the molecules catalogued in the International Nomenclature of Cosmetic Ingredients (INCI).

According to one embodiment, the targeting ligand of a lipid nanoparticle is palmitoyl pentapeptide-3 (or palmitoyl-KTTKS) or asiaticoside.

According to another embodiment, the targeting ligand of an aqueous nanoparticle is asiaticoside or modified hyaluronic acid modified (lipidised) by caproic acid (Teneliderm®).

This invention additionally concerns a method for preparing a nanoparticle according to the invention, comprising the following steps:
preparation of a lipid phase and an aqueous phase, whereby at least one of the two phases comprises a surfactant, at least one of the phases comprises a targeting ligand, and at least one of the two phases, if applicable, comprises an active ingredient;
emulsification of the lipid phase and the aqueous phase, resulting in the formation of nanoparticles, and
recovery of the nanoparticles formed.

The lipid phase and the aqueous phase are prepared by simply mixing the various components for each of the phases.

The active ingredient may be incorporated into one or both phases.

The targeting ligand is incorporated into one or both phases and, due to its amphiphilic character, positions itself at the interface between the internal and external membranes. Its lipophilic part is thus located in the lipid phase (L₂) or (L′₂), and its hydrophilic part is located in the aqueous phase (A₂) or (A′₂).

In the case of a lipid nanoparticle, the length of the hydrophilic part B is such that the end of the targeting ligand is located in the external membrane and not beyond the surface of this membrane.

More specifically, in the case of a lipid nanoparticle, the preparation of the lipid phase comprises, in particular, the incorporation of the components of the core that will form the lipid phase (L₁). The surfactant is included within the phase in which it is the most soluble. Typically, a water-soluble surfactant is incorporated into the aqueous phase and a lipid-soluble surfactant is incorporated into the lipid phase. The targeting ligand and the active ingredient are also incorporated into one or the other of the two phases based on their mainly hydrophilic or lipophilic character.

The emulsification step, which includes the mixing of these two phases, allows the various components to position themselves in order to form the core and the internal and external membranes of the lipid nanoparticle. In particular, the hydrophilic parts of the surfactant and the targeting ligand are positioned in the aqueous phase (A₂), which will constitute the external membrane, and their lipophilic parts are positioned in the lipid phase (L₂), which will constitute the internal membrane.

Preferably, the ligand is not grafted, coupled, conjugated, or bonded in any way with another compound at the time of its incorporation into one or both phases.

The method according to the invention thus does not comprise a step of grafting the targeting ligand, whether before or after the emulsification step. There are no impurities formed in the method, and there is no need for an additional purification step to obtain the lipid nanoparticles.

This method is thus simpler and less expensive to implement than prior-art methods.

According to one embodiment, the emulsification step is preceded by a pre-emulsification step comprising mixing the aqueous and lipid phases by mechanical agitation.

This pre-emulsification step consists of grossly mixing the lipid and aqueous phases by mechanical agitation, e.g., using a rotor-stator agitator.

It allows for a first rapid emulsification, resulting in a nearly homogeneous dispersion. The absence of solids and/or semi-solids greater in size than the millimetre scale is evaluated visually.

Preferably, the step of emulsifying the two phases is carried out by a high-energy method selected from sonication, high-pressure homogenisation (pressure applied between 100 and 200 Pa, advantageously between 500 and 1500 Pa) and microfluidisation.

Sonication consists of using ultrasound, generally using an ultrasound bath, to agitate the particles of a sample, e.g., to break molecular aggregates or cellular membranes, and allows, in particular, for reductions in the size of the particles. To obtain nano scale particles, more powerful sonicators must generally be used, such as Hielsher or Ultrasounics sonotrode sonicators.

High-pressure homogenisation consists of subjecting particles to the effects of pressure changes, acceleration, shearing, and impact, resulting in the reduction of their size.

Microfluidisation consists of using high pressure to force a fluid to enter microchannels having a specific configuration and to generate emulsification therein by a mechanism combining the effects of cavitation, shearing, and impact.

This invention additionally concerns the use of a lipid nanoparticle or aqueous nanoparticle according to the invention to vectorise one or more active ingredients, in particular cosmetics, dermatological pharmaceuticals, or pharmaceuticals.

In this description, ‘vectorisation of active ingredients’ refers to the encapsulation and delivery of active ingredients by a biocompatible vehicle captured by a target on which the active ingredient is to act biologically.’

This invention additionally concerns the use of a lipid nanoparticle or aqueous nanoparticle according to the invention for the preparation of a cosmetic, dermatological pharmaceutical, or pharmaceutical.

More specifically, it concerns the use of a lipid nanoparticle or aqueous nanoparticle according to the invention for the preparation of pharmaceutical composition for topical application.

Lastly, this invention concerns a cosmetic, dermatological pharmaceutical, or pharmaceutical composition comprising at least one lipid nanoparticle or aqueous nanoparticle according to the invention in combination with a cosmetically, dermatologically, or pharmaceutically acceptable vehicle.

More specifically, it concerns a cosmetic composition comprising at least one lipid nanoparticle or aqueous nanoparticle according to the invention, in combination, if applicable, with a cosmetically acceptable vehicle.

More specifically, it concerns a pharmaceutical composition comprising at least one lipid nanoparticle or aqueous nanoparticle according to the invention, in combination, if applicable, with a pharmaceutically acceptable vehicle.

The invention will be better understood based on the following description, provided by way of example only, referring to the attached drawings, in which:

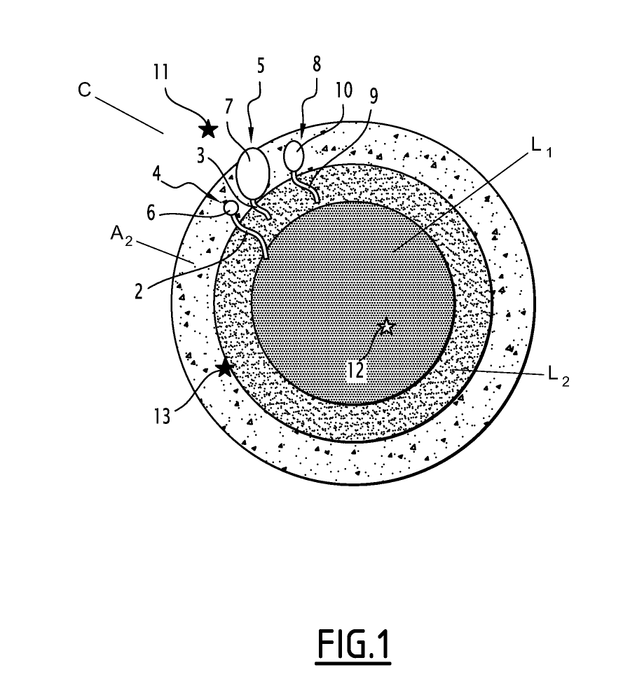

FIG. 1 is a large-scale section along a median vertical plane of a direct nanoemulsion comprising a lipid nanoparticle according to the invention and three different active ingredients;

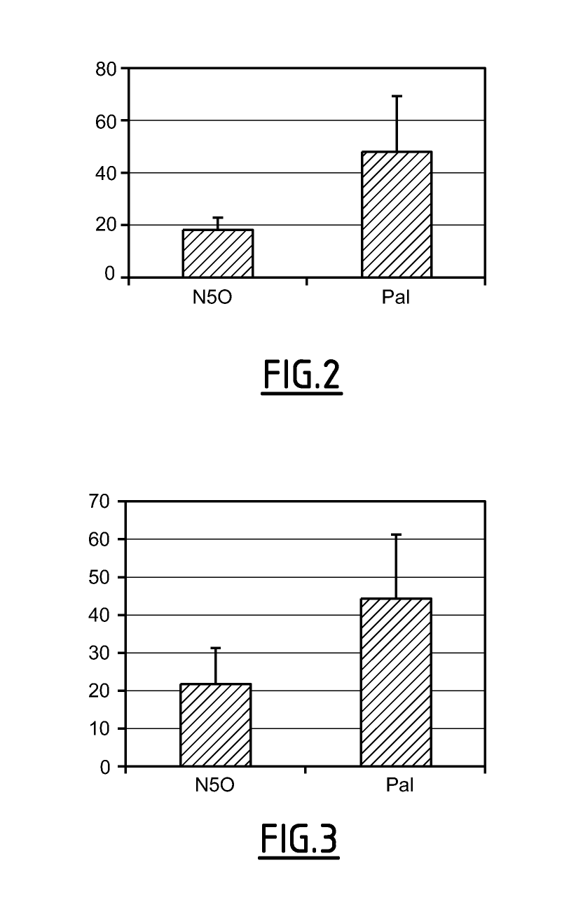

FIG. 2 is a graph showing the fluorescence intensity emitted per cell by a 3T3 fibroblastic cell line in the presence of nanoemulsions without a targeting ligand, referred to as N50, and nanoemulsions according to the invention having palmitoyl-KTTKS, referred to as Pal;

FIG. 3 is a graph showing the fluorescence intensity emitted per cell by a HaCat keratinocyte cell line in the presence of N50 and Pal nanoemulsions;

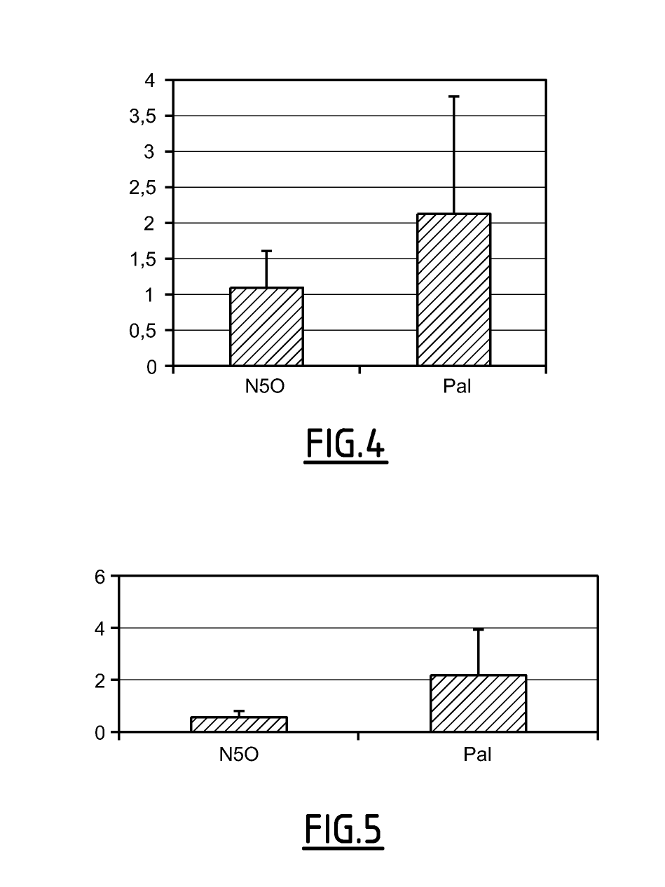

FIG. 4 is a graph showing the fluorescence intensity emitted per cell by human primary fibroblastic cells in the presence of N50 and Pal nanoemulsions; and

FIG. 5 is a graph showing the fluorescence intensity emitted per cell by human primary melanocytic cells in the presence of N50 and Pal nanoemulsions.

In FIG. 1, a lipid nanoparticle according to the invention comprises:
a core consisting of a lipid phase (L₁):
an internal membrane constituting a lipid phase (L₂) and comprising the lipophilic parts 2 and 3 of the lipid-soluble surfactant 4 and the water-soluble surfactant 5, respectively;
an external membrane constituting an aqueous phase (A₂) and comprising the hydrophilic parts 6 and 7 of the lipid-soluble surfactant 4 and the water-soluble surfactant 5, respectively; and
a targeting ligand 8 positioned such that its lipophilic part 9 is in the lipid phase (L₂) and its hydrophilic part 10 is in the aqueous phase (A₂).

Three active ingredients are incorporated into the nanoemulsion containing the lipid nanoparticle: The hydrophilic active ingredient 11 is in the continuous aqueous phase C, the lipophilic active ingredient 12 is in the lipid phase (L₁), and the amphiphilic active ingredient 13 is at the interface of the lipid (L₂) and aqueous (A₂) phases.

### EXAMPLES

### Example 1

### Preparation of Direct Targeting Nanoemulsions by Means of Palmitoyl-KTTKS

The table below indicates the composition of the aqueous and lipid phases of the nanoemulsions:

| | | | | | % mass |
|---|---|---|---|---|---|
| | | | | Mass | in final |
| | Compound | Trade name | Supplier | (g) | product |
| Aque- | Water | — | — | 375.00 | 75.00 |
| ous | PEG 40 | Myrj S40 | Croda | 55.00 | 11.00 |
| Phase | stearate | | | | |
| Lipid | Phospholipids | Phospholipon | Lipoid | 11.25 | 2.25 |
| Phase | Olive oil | | | 28.75 | 5.75 |
| | Wax | Lipocire | Gattefossé | 28.75 | 5.75 |
| | Palmitoyl- | — | Creative | 1.25 | 0.25 |
| | KTTKS | | Peptide | | |

A. Preparation of the Aqueous Phase

The aqueous phase was prepared by solubilising the surfactant Myrj S40, which was previously weighed, in water by agitating the dispersion with a magnetic agitator at 200 rpm for 10 min, at 45° C.

B. Preparation of the Lipid Phase

The lipid phase was prepared by heating the mixture of oil with Lipocire (solid lipids) and Phospholipon until the complete dissolution of the wax and phospholipids. The targeting ligand, palmitoyl-KTTKS, was then added, and the dispersion was mixed by means of a magnetic agitator at 200 rpm for 15 min at 45° C. until a homogeneous translucent solution was obtained.

C. Pre-Emulsification with Phase Mixture

The aqueous phase was added to the lipid phase. They were mixed together by means of an Ultra Turrax T25 (IKA Labortechnik) agitator at 20% of maximum power for 5 min until a milky, nearly homogeneous dispersion was obtained. The absence of solids and/or semi-solids greater in size than the millimetre scale was evaluated visually.

D. Preparation of the Targeting Nanoemulsions by Ultrasonication

The gross emulsion previously obtained was then ultrasonicated. More specifically, the gross emulsion was divided into five parts, each of which was poured into a 100 ml beaker. The sonotrode of the ultrasound probe (AV505 Ultrasonic processor, SONICS with a 3 mm bicylindrical sonotrode) was inserted into the first beaker, and the emulsion was subjected to sonication cycles (10 s ON/30 s OFF) for 20 min at 25% of maximum power. The beaker was placed in a water bath at room temperature during sonication in order to avoid any excessive increase in temperature that might degrade heat-sensitive molecules such as the targeting ligand, the active ingredients, or the preservatives.

The nanoemulsions thus prepared have an average size of 50 nm. The polydispersity index is 0.170.

The size and polydispersity of the nanoemulsion populations were measured by light diffusion on a Zeta Sizer Nano ZS (Malvern Instrument). A nanoemulsion sample was diluted to 0.1% in pure water and placed in a basin. The basin was then placed in the instrument, and three intensity measurements were obtained.

### Example 2

### Preparation of Direct Targeting Nanoemulsions by Means of Centella asiatica

The table below indicates the composition of the aqueous and lipid phases of the nanoemulsions:

| | | | | % mass |
|---|---|---|---|---|
| | | | Mass | in final |
| | Compound | Trade name | (kg) | product |
| Aque- | Water | — | 16.806 | 84.031 |
| ous | PEG 40 stearate | Myrj s40 | 0.445 | 2.224 |
| Phase | 1,2-hexanediol | KMO-6 | 0.100 | 0.500 |
| | Caprylyl glycol | | 0.040 | 0.200 |
| | Butylene glycol | | 2.000 | 10.000 |
| | Sodium taurate | Seppinoiv | 0.040 | 0.200 |
| | hydroxyethyl/ | EMT10 | | |
| | acryloyldimethyl | | | |
| | acrylate copolymer | | | |
| | EDTA | | 0.010 | 0.050 |
| Lipid | Lecithin | Phospholipon | 0.090 | 0.449 |
| Phase | Olive oil | | 0.229 | 1.145 |
| | Hydrogenated palm oil | Lipocire | 0.229 | 1.145 |
| | Melia Azadirachta extract | Nimbin | 0.001 | 0.005 |
| | Centella Asiatica leaf | | 0.005 | 0.025 |
| | extract | | | |
| | Tocopheryl acetate | Vitamine E | 0.005 | 0.025 |
| | | acétate | | |

A. Preparation of the Aqueous Phase

The aqueous phase was prepared by solubilising the surfactant Myrj S40 and the preservatives, which were previously weighed, in water by agitating the dispersion with a paddle agitator at 800 rpm for 30 min, at 45° C.

B. Preparation of the Lipid Phase

The lipid phase was prepared by heating the mixture of oil with Lipocire (solid lipids) and Phospholipon (phospholipids) until the complete dissolution of the wax and phospholipids. The active ingredient (Nimbin), the targeting ligand (Centella asiatica), and the antioxidant (vitamin E acetate) were then added, and the dispersion was mixed by means of a paddle agitator at 800 rpm for 45 min at 45° C. until a homogeneous translucent solution was obtained.

C. Pre-Emulsification with Phase Mixture

The aqueous phase was added to the lipid phase. They were mixed together by means of a rotor-stator agitator (Greerko) at 60% of maximum power for 20 min for 5 L until a milky, nearly homogeneous dispersion was obtained. The absence of solids and/or semi-solids greater in size than the millimetre scale was evaluated visually.

D. Preparation of the Targeting Nanoemulsions by High-Pressure Homogenisation

The gross emulsion previously obtained was then passed through the homogeniser (Panda Plus, GEA NIRO SOAVI) for 4 h in order to reduce the size of the droplets of the emulsion. More specifically, the gross emulsion was inserted into the reservoir of the device under agitation to avoid creaming of the gross emulsion and under temperature control (T=45° C.±5° C.) by means of a water-based heat exchanger to avoid excessive increases in the temperature of the emulsion, which might result in the degradation of certain heat-sensitive molecules such as the targeting ligand, the active ingredients, or the preservatives. The pressure was set at 1000 bar.

The nanoemulsions thus prepared have an average size of 80 nm. The polydispersity index is 0.180.

The size and polydispersity of the nanoemulsion populations were measured by light diffusion on a Zeta Sizer Nano ZS (Malvern Instrument). A nanoemulsion sample was diluted to 0.1% in pure water and placed in a basin. The basin was then placed in the instrument, and three intensity measurements were obtained.

### Example 3

### Preparation of Inverse Targeting Nanoemulsions by Means of Centella asiatica

The table below indicates the composition of the aqueous and lipid phases of the nanoemulsions:

| | | Mass | % mass in |
|---|---|---|---|
| | Compound | (mg) | final product |
| Aqueous | Demineralised water | 0.119 | 5.95 |
| Phase | Glycerol | 0.050 | 2.50 |
| | Tris | 0.010 | 0.50 |
| | Sodium chloride | 0.001 | 0.05 |
| | Asiaticoside | 0.005 | 0.25 |
| Lipid | Parleam | 1.565 | 78.25 |
| Phase | Arlacel P135 | 0.200 | 10.00 |
| | Diisostearic plurol | 0.050 | 2.50 |

In an appropriate receptacle, the oil phase was prepared by homogenisation of the oil and the stabiliser at 50° C.

In a second appropriate receptacle, the aqueous phase was prepared by homogenisation at room temperature of any additives in water, as well as the various optional hydrophilic adjuvants (osmotic, thickener, preservative . . . ).

The aqueous phase is added to the lipid phase either manually or by magnetic or turbine agitation. The two phases are grossly mixed, and then the mixture is homogenised by ultrasound using devices such as the AV505® sonicator (Sonics, Newtown) for volumes less than 200 g or the IUP 1000hd (Hielsher, Germany) for greater volumes. During the sonication, the receptacle containing the dispersion is thermostated.
The nanoemulsions thus prepared have an average size less than 50 nm. They are stable and transparent.
The size of the nanoemulsion populations was measured by quasi-elastic light diffusion on a Zeta Sizer Nano ZS (Malvern Instrument).

### Example 4

### Preparation of Inverse Targeting Nanoemulsions by Means of Teneliderm®

The table below indicates the composition of the aqueous and lipid phases of the nanoemulsions:

| | | Mass | % by |
|---|---|---|---|
| | Compounds | (mg) | mass |
| Dispersed | Demineralised water (dispersed phase) | 0.637 | 12.74 |
| Phase | Sodium chloride (osmotic agent) | 0.0065 | 0.13 |
| | Hyacare 50 (osmotic agent) | 0.0065 | 0.13 |
| Continous | Phytosqualane (oil - continuous phase) | 1.5 | 30 |
| Phase | Luvitol (oil - continuous phase) | 2.1 | 42 |
| | Cithrol dphs-SO-(MV) (surfactant) | 0.6245 | 12.49 |
| | Dub Iso G3 (surfactant) | 0.125 | 2.5 |
| | Teneliderm ® (targeting ligand/active) | 0.0005 | 0.01 |

Teneliderm® is the targeting ligand; it is a hyaluronic acid lipidised with caproic acid. CD44 is a transmembrane receptor for glycosaminoglycans, including hyaluronic acid, with which it has a significant affinity. Teneliderm® is inserted into the fat phase.

In two appropriate receptacles, the oil (continuous) and aqueous (dispersed) phases were prepared separately and heated to 50° C.

The dispersed phase is added manually to the continuous phase. The two phases are grossly mixed, and then the mixture is homogenised by ultrasound using devices such as the AV505® sonicator (Sonics, Newtown) for volumes less than 200 g.

The power delivered is 25%; the sonication time is 5 min (Pulse on: 10 s, pulse off: 30 s). The number of joules delivered is 37500 J.

During the sonication, the receptacle containing the dispersion is immersed in a water bath. The average diameter of the dispersed phase is determined by quasi-elastic light diffusion on a Zeta Sizer Nano ZS (Malvern Instrument). The sizes obtained are less than 150 nm.

### Example 5

### Preparation of Inverse Targeting Nanoemulsions by Means of Teneliderm®

The table below indicates the composition of the aqueous and lipid phases of the nanoemulsions:

| | | Mass | % by |
|---|---|---|---|
| | Compounds | (mg) | mass |
| Dispersed | Demineralised water (dispersed phase) | 0.637 | 12.74 |
| Phase | Sodium chloride (osmotic agent) | 0.0065 | 0.13 |
| | Hyacare 50 (osmotic agent) | 0.0065 | 0.13 |
| Continous | Phytosqualane (oil - continuous phase) | 1.5 | 30 |
| Phase | Luvitol (oil - continuous phase) | 2.1 | 42 |
| | Cithrol dphs-SO-(MV) (surfactant) | 0.62 | 12.4 |
| | Dub Iso G3 (surfactant) | 0.125 | 2.5 |
| | Teneliderm ® (targeting ligand/active) | 0.005 | 0.1 |

Teneliderm® is the targeting ligand; it is a hyaluronic acid lipidised with caproic acid. CD44 is a transmembrane receptor for glycosaminoglycans, including hyaluronic acid, with which it has a significant affinity. Teneliderm® is inserted into the fat phase.

In two appropriate receptacles, the oil (continuous) and aqueous (dispersed) phases were prepared separately and heated to 50° C.

The dispersed phase is added manually to the continuous phase. The two phases are grossly mixed, and then the mixture is homogenised by ultrasound using devices such as the AV505® sonicator (Sonics, Newtown) for volumes less than 200 g.

The power delivered is 25%; the sonication time is 5 min (Pulse on: 10 s, pulse off: 30 s). The number of joules delivered is 37500 J.

During the sonication, the receptacle containing the dispersion is immersed in a water bath. The average diameter of the dispersed phase is determined by quasi-elastic light diffusion on a Zeta Sizer Nano ZS (Malvern Instrument). The sizes obtained are less than 150 nm.

### Example 6

### Evaluation of Direct Targeting Nanoemulsions by Means of Palmitoyl-KTTKS

In order to evaluate the targeting capability of the nanoemulsions of Example 1, identical nanoemulsions were prepared on laboratory scale (smaller volumes), and fluorophores (Dil) were incorporated in order to carry out fluorescence measurements.

### Preparation of the Nanoemulsions

The table below indicates the composition of the aqueous and lipid phases of the nanoemulsions:

| | | | | | % mass |
|---|---|---|---|---|---|
| | | | | Mass | in final |
| | Compound | Trade name | Supplier | (mg) | product |
| Aque- | Water | — | — | 1500 | 75.00 |
| ous | PEG 40 stearate | Myrj s40 | Croda | 215 | 10.75 |
| Phase | | | | | |
| Lipid | Phospholipids | Phospholipon | Lipoid | 45 | 2.25 |
| Phase | Olive oil | | | 115 | 5.75 |
| | Wax | Lipocire | Gattefossé | 115 | 5.75 |
| | Palmitoyl- | — | Creative | 10 | 0.50 |
| | KTTKS | | Peptide | | |

A. Preparation of the Aqueous Phase

The aqueous phase was prepared by solubilising the surfactant Myrj S40, dissolved in phosphate-buffered saline (PBS) 1× in water.

B. Preparation of the Lipid Phase

The lipid phase was prepared by mixing soya oil (Soybean oil, Sigma Aldrich), paraffin (Semi-synthetic glycerides, Suppocire NC, Gattefossé, France), soybean phospholipids (Phospholipon 75, Lipoid, Germany) and 0.1% by mass of the fluorophore Dil (1,1′-Dioctadecyl-3,3,3′,3′-tetramethylindocarbocyanine perchlorate, Sigma Aldrich). The lipid phase thus prepared contains 16% by mass of phospholipids and 84% by mass of lipids.

C. Preparation of the Targeting Nanoemulsions by Ultrasonication

20% of the lipid phase was dispersed in 80% of the aqueous phase, resulting in a mixture with a ratio of phospholipids/Myrj S40 of 0.18 and a ratio of Myrj S40/(oil+wax) of 0.55.

The mixture was then emulsified with a 3 mm ultrasound probe following sonication cycles (10 s ON/30 s OFF) for 10 min.

The nanoemulsions thus prepared have an average size of 50 nm.

The nanoemulsion suspensions were then dialysed against 500 ml PBX 1× for one night. Then, they were recovered, diluted to a content of 10% by mass, filtered through 0.2 μm pores, and then stored at 4° C. until they were used.

### Evaluation of Targeting Capability

The targeting capability of the nanoemulsions was evaluated by comparing their adhesion to various cells targeted by palmitoyl-KTTKS and that of simple nanoemulsions of the same size without any targeting ligand.

Adhesion was evaluated by fluorescence measurements on cells, thus allowing for quantification of the interaction between nanoemulsions and cells.

More specifically, the adhesion of the nanoemulsions was tested on a 3T3 fibroblastic cell line, a HaCaT keratinocyte cell line, primary human melanocytes, and primary human fibroblasts.

A. Cell Culture

The cells and reagents used for the cell culture were supplied by Life Technologies (Villebon sur Yvette, France). Human dermal fibroblasts (HDFa) from a 37-year-old woman, keratinocytes from a HaCaT cell line provided by the Deutsches Krebsforschungszentrum (Cell Line Service, Eppelheim, Germany), were cultivated in Dulbecco's Modified Eagle Medium (DMEM), with 10% by volume of heat-deactivated foetal bovine serum, 50 UI/ml penicillin, and 50 μg/mL streptomycin. The cells were incubated at 37° C. in an atmosphere of 5% CO₂, saturated with humidity.

The HaCaT passes were carried out before the cells reached 100% confluence. More specifically, the culture medium was removed from the culture containers; the cells were then washed with PBS 1× containing neither calcium nor magnesium, then 2 ml of a trypsin/EDTA solution were added and the containers were placed in the incubator for 3 min. They were kept at room temperature until the cells became round and detached from the bottom of the containers. DMEM-FCS solution was added in order to inhibit the activity of trypsin, and the remaining cells were removed by grinding. The cells were centrifuged for 7 min at 300 g (g=9.81 m·s⁻²), and the ball obtained was suspended in 1 mL DMEM-FCS for numbering and seeding.

The HDFa passages were carried out when the cells reached 80-90% confluence, as with the keratinocytes. On the other hand, the trypsin activity was inhibited by adding to the cell solution an equal volume of purified soybean solution, a trypsin inhibitor.

B. Evaluation of Cellular Adsorption of the Nanoemulsions

The capacity of the nanoemulsions to be adsorbed on cells was evaluated as a function of the quantity of ligand encapsulated. The cells were seeded in eight chambers positioned on a LabTek glass microscope slide (Fisher Scientific, Illkirsh, France) and placed in an incubator for 48 h for recovery of the cell culture after the passes. The culture medium was then replaced with 250 μg/ml nanoemulsion suspension and incubated for 1 h at 37%, at 5% CO₂. The cells were then washed twice with 200 μL PBS 1× for 10 min, affixed with 200 μL of a 4%(w/v) paraformaldehyde solution in PBS 1× for 10 min, and finally washed with 200 μL PBS 1×. Lastly, the glass slide was separated from the plastic chambers and mounted with Fluoroshield™ with DAPI (Sigma-Aldrich, St Quentin Fallavier, France) to observe the flourescence microscopically (Nikon Eclipse E600) equipped with Dil filters (G2A filters set, Ex 510-560 nm, DM 575 nm, BA 590 nm) (Nikon, Champigny sur Marne, France) and DAPI filters.

The optical and fluorescent images were recorded with a CCD camera (Cascade 512B, Photometrics, Tucson, Ariz., USA) using the MetaVue software (Molecular Devices, Roper Scientific, Evry, France) in an identical acquisition configuration (e.g., with a gain of 5 MHz and an exposure time of 100 ms) to allow for comparisons of the images.

The fluorescence intensity emitted per cell was measured for the various cell combinations prepared in the presence of N50 contron nanoemulsions without a targeting ligand and Pal nanoemulsions having palmitoyl-KTTS (FIG. 2-5).

The results shown in FIGS. 2-5 show that the adhesion of the targeting nanoemulsions is superior to that of the control nanoemulsions. The fluorescence intensity emitted in the case of the targeting nanoemulsions is at least twice as high as that measured in the case of the control nanoemulsions.

These observations thus show a cellular targeting efficacy due to the presence of the targeting ligand despite its position within the external membrane of the nanoemulsions.

1. Nanoparticle comprising:
a core consisting of a lipid phase (L₁) or an aqueous phase (A₁);
at least one surfactant comprising a hydrophilic part and a lipophilic part;
an internal membrane surrounding the core;
an external membrane surrounding the internal membrane; and
at least one targeting ligand comprising a lipophilic part and a hydrophilic part;
in which:
when the core consists of a lipid phase (L₁):
the internal membrane constitutes a lipid phase (L₂) comprising the lipophilic part of the surfactant;
the external membrane constitutes an aqueous phase (A₂) comprising the hydrophilic part of the surfactant; and
the targeting ligand is such that its lipophilic part is in the lipid phase (L₂) and its hydrophilic part has a length that is less than the thickness of the external membrane in the aqueous phase (A₂);
when the core consists of an aqueous phase (A₁):
the internal membrane constitutes an aqueous phase (A′₂) comprising the hydrophilic part of the surfactant; and
the external membrane constitutes a lipid phase (L′₂) comprising the lipophilic part of the surfactant.

2. Nanoparticle according to claim 1, in which the external membrane that constitutes an aqueous phase (A₂) is between 1 and 7 nm in length, and the hydrophilic part of the targeting ligand located in the aqueous phase (A₂) has a length between 0.2 and 5 nm.

3. Nanoparticle according to claim 1, comprising at least one active ingredient.

4. Nanoparticle according to claim 1, having a diameter between 10 and 1000 nm.

5. Nanoparticle according to claim 1, in which the surfactants comprise polyethylene glycol (PEG) chains.

6. Nanoparticle according to claim 1, in which the targeting ligand is selected from compounds of formula (I):
A-Y-B (I)
in which:
A is the lipophilic part;
Y is a chemical group capable of linking A and B via covalent bonds; and
B is the hydrophilic part.

7. Nanoparticle according to claim 1, in which the targeting ligand is a sugar, biomolecule, polymer, or biopolymer.

8. Nanoparticle according to claim 1, in which the targeting ligand is palmitoyl pentapeptide-3, asiaticoside, or hyaluronic acid lipidised by caproic acid.

9. Nanoemulsion comprising at least one nanoparticle according to claim 1 and a continuous phase surrounding the nanoparticle.

10. Method for producing a nanoparticle according to claim 1, comprising the following steps:
preparation of a lipid phase and an aqueous phase, whereby at least one of the two phases comprises a surfactant, at least one of the phases comprises a targeting ligand;
emulsification of the lipid phase and the aqueous phase, resulting in the formation of nanoparticles, and
recovery of the nanoparticles formed.

11. (canceled)

12. Cosmetic, dermatological pharmaceutical, or pharmaceutical composition comprising at least one nanoparticle according to claim 1.
